(19) United States
(12) Patent Application Publication
(10) Pub. No.: US 2009/0171222 A1
(43) Pub. Date: Jul. 2, 2009

(54) APPARATUS AND METHOD FOR MONITORING BLOOD PRESSURE CUFF WEAR

(75) Inventor: Humberto Valdes, Tampa, FL (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/968,338

(22) Filed: Jan. 2, 2008

(51) Int. Cl. A61B 5/022

(52) U.S. Cl. 600/490

(57) ABSTRACT

A blood pressure cuff assembly is disclosed herein. The blood pressure cuff assembly includes a data storage device and cuff data stored on the data storage device. The cuff data pertains to a degree of wear of the pressure cuff assembly and/or an age of the pressure cuff assembly. A corresponding blood pressure monitoring system incorporating the blood pressure cuff assembly, and a method for implementing the blood pressure cuff assembly are also disclosed.

### BACKGROUND OF THE INVENTION

The subject matter disclosed herein relates to an apparatus and method for monitoring blood pressure cuff wear and blood pressure cuff age.

Conventional non-invasive blood pressure (NIBP) monitoring systems generally inflate a pressure cuff above the patient's systolic pressure and measure oscillations in the cuff as the cuff is deflated. The pressure cuff is wrapped around a limb and secured thereto with a fastening mechanism such as, for example, a hook-and-loop fastening mechanism. After wrapping and securing the pressure cuff to a limb, a cuff bladder is inflated with air in order to apply pressure. The oscillations monitored by the NIBP system are transmitted through the air contained within the cuff bladder during a blood pressure measurement.

One problem with conventional NIBP systems is that the pressure cuff can become worn after being subjected to numerous usage cycles. A worn pressure cuff may take longer to inflate and deflate thereby extending the requisite duration of the NIBP determination and correspondingly increasing patient discomfort. Excessively worn pressure cuffs may also diminish the precision of a blood pressure estimate. This problem can be exacerbated by the fact that pressure cuff wear is not always readily identifiable such that hospital personnel may inadvertently use worn pressure cuffs that should be discarded. Old pressure cuffs may suffer from some of the same problems identified with respect to excessively worn pressure cuffs.

### BRIEF DESCRIPTION OF THE INVENTION

The above-mentioned shortcomings, disadvantages and problems are addressed herein which will be understood by reading and understanding the following specification.

In an embodiment, a blood pressure cuff assembly includes a data storage device and cuff data stored on the data storage device. The cuff data pertains to a degree of wear of the pressure cuff assembly and/or an age of the pressure cuff assembly.

In another embodiment, a blood pressure monitoring system includes a pressure cuff assembly comprising a cuff, an inflatable bladder secured to and/or disposed within the cuff, and a data storage device secured to and/or disposed within the cuff. The data storage device is configured to store cuff data. The blood pressure monitoring system also includes a pressure monitor connected to the pressure cuff assembly. The pressure monitor includes a controller connected to the data storage device. The controller is configured to estimate a degree of wear of the pressure cuff assembly and/or an age of the pressure cuff assembly based on the cuff data stored on the data storage device.

In yet another embodiment, a method includes providing a pressure cuff assembly comprising a data storage device, and storing a variable on the data storage device. The method also includes incrementing the variable stored on the data storage device each time the pressure cuff assembly is implemented to measure a blood pressure such that the variable represents the number of pressure cuff assembly usage cycles. The method also includes communicating a warning if the variable stored on the data storage device exceeds a predetermined usage limit.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of a non-invasive blood pressure monitoring system attached to a patient in accordance with an embodiment;

FIG. 2 is a schematic diagram of a pressure cuff assembly in accordance with an embodiment; and

FIG. 3 is a schematic diagram of a pressure cuff assembly in accordance with another embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

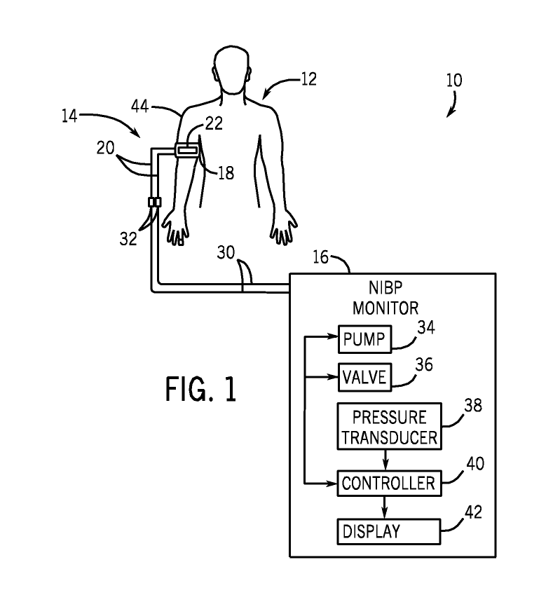

Referring to FIG. 1, a non-invasive blood pressure (NIBP) monitoring system 10 attached to a patient 12 is shown in accordance with an embodiment. The NIBP monitoring system 10 includes a pressure cuff assembly 14 that is connectable to a NIBP monitor 16. The NIBP monitoring system 10 may, for example, be implemented to estimate a patient's mean arterial pressure (MAP), systolic blood pressure (SBP), and/or diastolic blood pressure (DBP) using an Oscillometric method that is well known to those skilled in the art.

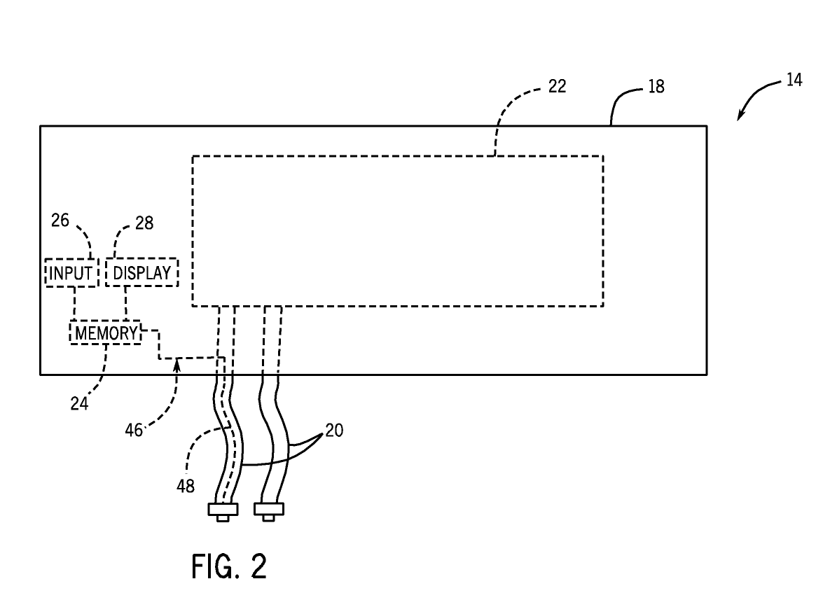

The pressure cuff assembly 14 includes a cuff 18, and one or more cuff tubes 20 that are pneumatically coupled with an inflatable bladder 22. As shown in FIG. 2, the pressure cuff assembly 14 also includes a data storage device such as the memory 24, and may optionally include an input device 26 and/or a display 28.

Referring again to FIG. 1, the NIBP monitor 16 includes one or more monitor tubes 30 adapted for connection to the cuff tubes 20 via the connectors 32. The connectors 32 may be disposed on the terminal end portions of the monitor tubes 30 and/or the cuff tubes 20 and are configured to form a pneumatic connection therebetween.

The NIBP monitor 16 also includes a pump 34 adapted to inflate the bladder 22, and one or more valves 36 adapted to deflate the bladder 22. The NIBP monitor 16 includes a pressure transducer 38 operable to sense pressure pulses at or near the point of cuff 18 attachment. A controller 40 may be implemented to convert the pressure pulse data from the pressure transducer 38 into blood pressure data. The blood pressure data from the controller 40 can be graphically conveyed via the display 42.

Referring to FIG. 2, a more detailed schematic illustration of the pressure cuff assembly 14 is shown in accordance with an embodiment. The cuff 18 is generally rectangular and is long enough to wrap around a limb 44 of the patient 12 (shown in FIG. 1). The limb 44 is depicted in FIG. 1 as comprising the patient's upper arm, however it should be appreciated that the cuff 18 may alternatively be applied to other locations (e.g., forearm) and other limbs.

According to the embodiment depicted in FIG. 2, the memory 24 is integrally retained within the material comprising the cuff 18. According to another embodiment, the memory 24 may be secured to a pressure cuff assembly component such as, for example, the cuff tubes 20. According to yet another embodiment, the memory 24 may comprise a discrete device adapted for attachment to a pressure cuff assembly.

The memory 24 may comprise a non-volatile data storage device so that recorded data is preserved even if the memory 24 loses power. According to one embodiment, a conductor 46 couples the memory 24 with the controller 40 of the NIBP monitor 16 (shown in FIG. 1). The conductor 46 may, for example, comprise a first portion 48 disposed within one of the cuff tubes 20 and a second portion (not shown) disposed within one of the monitor tubes 30 such that an electrical connection is formed when the cuff tubes 20 are coupled with the monitor tubes 30. According to another embodiment, the memory 24 may be wirelessly connected to the controller 40.

Referring to FIGS. 1 and 2, the memory 24 can be powered by the NIBP monitor 16 via the conductor 46, or by a dedicated power source such as a battery (not shown). The memory 24 is configured to store cuff data that may, for example, pertain to the degree of wear and/or the age of the pressure cuff assembly 14. According to one embodiment, the memory 24 stores cuff data comprising the number of pressure cuff usage cycles as an approximation of the pressure cuff's degree of wear. For purposes of this disclosure, the number of pressure cuff usage cycles should be defined to include the number of times a given pressure cuff assembly is implemented to measure blood pressure.

The following section will provide an exemplary method for recording cuff data in the form of pressure cuff usage cycles, and for implementing the recorded cuff data to identify and communicate excessive pressure cuff wear. Each time the controller 40 actuates the pump 34 in order to inflate the bladder 22 and thereby obtain an NIBP measurement, the controller 40 may also increment a variable representing the number of pressure cuff usage cycles and thereafter store the incremented variable on the memory 24 such that each pressure cuff assembly usage is recorded. The controller 40 can read the recorded cuff data on the memory 24 as soon as the pressure cuff assembly 14 is connected to the NIBP monitor 16. The controller 40 can then compare the number of pressure cuff usage cycles with a predetermined usage limit. If the number of pressure cuff usage cycles exceeds the predetermined usage limit, the controller 40 can transmit a visual warning via the display 42. Accordingly, a potential user is alerted to the fact that a given pressure cuff assembly may be excessively worn and should be discarded.

The memory 24 may optionally be implemented to store other types of data in addition to the previously described cuff data. For example, the memory 24 may be implemented to store patient data identifying a particular patient and/or medical data including the patient's blood pressure. In this manner, the pressure cuff assembly 14 could be coupled directly with a computer in order to download at least a portion of the patient's medical history.

The pressure cuff assembly 14 may optionally include the input device 26 and/or the display 28 that are each connected to the memory 24. The input device 26 may comprise a series of buttons allowing a user to retrieve cuff data or any other data stored on the memory 24. The data retrieved from the memory 24 may then be visually conveyed via the display 28. The optional components 26, 28 are particularly well suited to embodiments in which a battery powers the memory 24 because these components would allow a user to identify the pressure cuffs degree of wear and/or age in a manner that does not require a NIBP monitor.

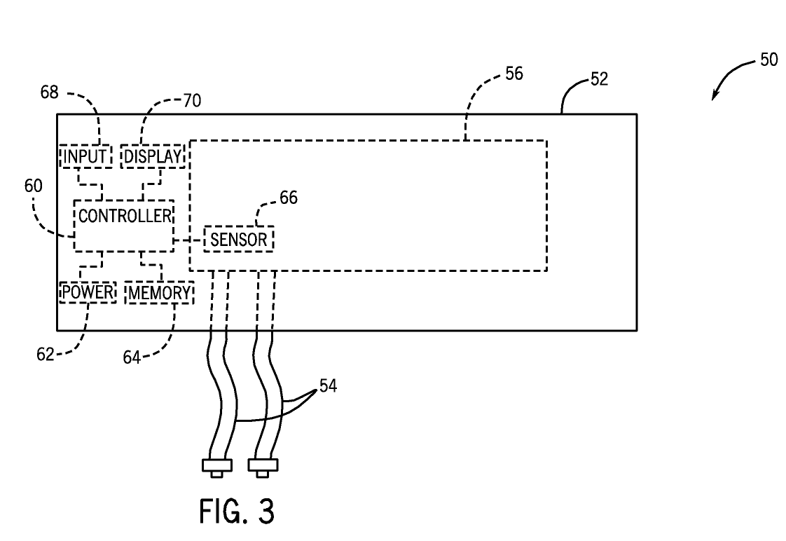

Referring to FIG. 3, a pressure cuff assembly 50 is shown in accordance with an embodiment. The pressure cuff assembly 50 includes a cuff 52, and one or more cuff tubes 54 pneumatically coupled with and an inflatable bladder 56. The pressure cuff assembly 50 is connectable to a NIBP monitor via the cuff tubes 54, and is thereafter operable to measure a patient's blood pressure in a manner similar to that described hereinabove with respect to the pressure cuff assembly 14 (shown in FIG. 2).

The pressure cuff assembly 50 includes a controller 60 that can be secured to the cuff 52. The controller 60 is powered by a power supply 62 that may, for example, include a battery. The pressure cuff assembly 50 also includes a data storage device such as the memory 64 that is electronically coupled with the controller 60. The memory 64 may comprise a non-volatile data storage device so that recorded data is preserved even if the memory 64 loses power. The memory 64 is configured to store cuff data that may, for example, pertain to the degree of wear and/or the age of the pressure cuff assembly 50. The memory 64 may also be implemented to store other types of data including patient data and/or medical data as was described with respect to the memory 24 (shown in FIG. 2).

The pressure cuff assembly 50 includes a sensor 66 that is electronically coupled with the controller 60. According to one embodiment, the sensor 66 may comprise a strain gage secured to a surface of the bladder 56 and adapted to sense bladder expansion. According to another embodiment, the sensor 66 may comprise a pressure sensor disposed within the bladder 56 and adapted to sense bladder pressure.

The controller 60 is adapted to identify bladder 56 inflation based on feedback from the sensor 66. As an example, the controller 60 may increment a variable stored on the memory 64 each time the bladder 56 is inflated. In this manner, the memory 64 can store cuff data in the form of a bladder inflation count that represents the degree of pressure cuff assembly wear. At startup, the controller 60 can read the bladder inflation count stored on the memory 64 and compare it with a predetermined usage limit. If the number of bladder inflations exceeds the predetermined usage limit, the controller 32 can transmit a warning adapted to avoid the unintentional use of an excessively worn cuff.

The pressure cuff assembly 50 may optionally include an input device 68 and a display 70. The input device 68 may comprise a series of buttons allowing a user to retrieve cuff data or other data stored on the memory 64. The data retrieved from the memory 64 may then be visually conveyed via the display 70. Accordingly, a user can readily identify the age or degree of wear of a given pressure cuff assembly in a manner that does not require a NIBP monitor.

The memory 24 of FIG. 2 and the memory 64 of FIG. 3 have been described in accordance with an embodiment as being adapted to store cuff data in the form of usage cycles or a bladder inflation count. This embodiment assumes that cuff wear is related to the number of times a given cuff is implemented to provide a NIBP estimate. It should be appreciated that the memories 24 and 64 may alternatively store other types of cuff data related to cuff wear or cuff age. For example, the memories 24 and 64 could store cuff data pertaining to the date the cuff was manufactured so that cuff assemblies deemed excessively old could be easily identified and discarded.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

I claim:

1. A blood pressure cuff assembly comprising:
a data storage device; and
a cuff data stored on the data storage device, said cuff data pertaining to a degree of wear of the pressure cuff assembly and/or an age of the pressure cuff assembly.

2. The blood pressure cuff assembly of claim 1, wherein the data storage device comprises a non-volatile memory device.

3. The blood pressure cuff assembly of claim 1, wherein the cuff data comprises an estimate of the number of times the blood pressure cuff assembly has been implemented to measure a blood pressure.

4. The blood pressure cuff assembly of claim 1, wherein the cuff data comprises a blood pressure cuff assembly manufacturing date.

5. The blood pressure cuff assembly of claim 1, further comprising a patient data and/or a medical data stored on the data storage device.

6. The blood pressure cuff assembly of claim 1, further comprising a display operatively connected to the data storage device, said display being configured to visually convey the cuff data.

7. The blood pressure cuff assembly of claim 1, further comprising an input device operatively connected to the data storage device.

8. The blood pressure cuff assembly of claim 1, further comprising a controller operatively connected to the data storage device.

9. The blood pressure cuff assembly of claim 8, further comprising a sensor operatively connected to the controller.

10. A blood pressure monitoring system comprising:
a pressure cuff assembly comprising:
a cuff;
an inflatable bladder secured to and/or disposed within the cuff; and
a data storage device secured to and/or disposed within the cuff, said data storage device configured to store cuff data; and
a pressure monitor connected to the pressure cuff assembly, the pressure monitor including a controller connected to the data storage device, the controller being configured to estimate a degree of wear of the pressure cuff assembly and/or an age of the pressure cuff assembly based on the cuff data stored on the data storage device.

11. The blood pressure monitoring system of claim 10, wherein the data storage device comprises a non-volatile memory device.

12. The blood pressure monitoring system of claim 10, further comprising a conductor electrically coupling the data storage device with the controller.

13. The blood pressure monitoring system of claim 12, wherein the conductor is disposed at least partially within a tube pneumatically coupling the pressure cuff assembly with the pressure monitor.

14. The blood pressure monitoring system of claim 10, wherein the pressure monitor further includes a display adapted to visually convey the estimated degree of wear of the pressure cuff assembly and/or the estimated age of the pressure cuff assembly.

15. A method comprising:
providing a pressure cuff assembly comprising a data storage device;
storing a variable on the data storage device;
incrementing the variable stored on the data storage device each time the pressure cuff assembly is implemented to measure a blood pressure such that the variable represents the number of pressure cuff assembly usage cycles; and
communicating a warning if the variable stored on the data storage device exceeds a predetermined usage limit.

16. The method of claim 15, wherein said communicating a warning comprises visually communicating a warning via a display.

17. The method of claim 15, further comprising storing a pressure cuff assembly manufacturing date on the data storage device.

18. The method of claim 15, further comprising storing a patient data on the data storage device.

19. The method of claim 15, further comprising storing a medical data on the data storage device.
